(19) United States
(12) Patent Application Publication
(10) Pub. No.: US 2005/0031668 A1
(43) Pub. Date: Feb. 10, 2005

(54) Implantable polymeric device for sustained release of nalmefene

(76) Inventors: Rajesh A. Patel, Redwood City, CA (US); Louis R. Bucalo, San Francisco, CA (US); Lauren Costantini, San Francisco, CA (US)

Correspondence Address: MORRISON & FOERSTER LLP, 755 PAGE MILL RD, PALO ALTO, CA, 94304-1018, US

(21) Appl. No.: 10/856,178

(22) Filed: May 27, 2004

Related U.S. Application Data
(60) Provisional application No. 60/474,916, filed on May 30, 2003.

(51) Int. Cl. A61K 31/485

(52) U.S. Cl. 424/426; 514/282

(57) ABSTRACT

The present invention provides compositions, methods, and kits for administration of nalmefene for treatment of alcoholism, nicotine dependence, or another condition for which treatment with nalmefene is therapeutically beneficial. The invention provides a biocompatible nonerodible polymeric device which releases nalmefene continuously with generally linear release kinetics for extended periods of time. Nalmefene is released through pores that open to the surface of the polymeric matrix in which it is encapsulated. The device may be administered subcutaneously to an individual in need of continuous treatment with nalmefene.

### CROSS-REFERENCE TO RELATED APPLICATIONS

This application claims the benefit of U.S. provisional application No. 60/474,916, filed May 30, 2003, which is incorporated herein by reference in its entirety.

### TECHNICAL FIELD

The invention provides a nonbioerodible, polymeric device for subcutaneous implantation and sustained release of nalmefene for treatment of alcoholism, nicotine dependence, or another condition for which nalmefene administration is therapeutically beneficial.

### BACKGROUND OF THE INVENTION

In the U.S., 14 million people suffer from alcohol dependency or met diagnostic criteria for alcohol abuse disorder (NIAAA statistics). Available treatment methods for alcohol dependence include brief intervention, behavioral and cognitive-behavioral approaches, psychosocial and motivation-enhancement methods, and pharmacotherapies. Most alcoholics initially achieve a period of sobriety with or without formal treatment. However, many return to drinking within a short period of time. Thus, alcoholism is a chronic relapsing disorder. The first months following cessation of drinking show the highest risk for relapse and offer the greatest opportunity for pharmacological intervention. However, success with pharmacotherapy is often limited by poor patient compliance, variability in blood levels of the drug, and adverse effects associated with drug toxicity at the doses required for clinical efficacy. A long-term delivery system would improve upon several aspects of pharmacotherapy for alcohol dependence.

Aversive therapy with disulfiram (Antabuse) was the only pharmacological treatment for alcohol dependence available in the U.S. for many years. However, therapy with this drug suffered from high rates of severe adverse reactions, drinking relapse, and medication noncompliance. (Fuller et al. (1986) JAMA 256:1449-55) Naltrexone was approved in 1994 as a nonaversive prescription drug for alcohol dependence. (Croop et al. (1997) Arch Gen Psychiatry 54(12):1130-35; O'Malley et al. (1992) Arch Gen Psychiatry 49(11):881-87; Volpicelli et al. Arch Gen Psychiatry 49(11):876-80) Reduced risk of relapse to heavy drinking is observed among those who are highly compliant with treatment. (O'Malley et al. (1996) Arch Gen Psychiatry 53(3):217-24; Oslin et al. (1997) Am J Geriatr Psychiatry 5(4):324-32; Volpicelli et al. (1997) Arch Gen Psychiatry 54(8):737-42) Use of naltrexone has certain limitations, including intolerable nausea (Croop et al., supra) and dose-dependent hepatotoxic side effects. Thus, this medication is contraindicated in alcoholic patients with liver disease. (Physicians' Desk Reference 1997; 51st edition: 957-59)

Nalmefene is a pure opioid antagonist structurally similar to naltrexone, and is approved in the U.S. for reversal of effects of opioids and the management of opioid overdose (nalmefene hydrochloride; Revex®). Nalmefene has no agonist activity and thus no abuse potential (Fudala et al. (1991) Clin Pharmacol Ther 49(3):300-306), a longer half-life (Dixon et al. (1986) Clin Pharmacol Ther 39(1):49-53), and no serious adverse effects such as respiratory depression or hepatotoxicity.

Nalmefene has been shown to be effective in animal models of alcoholism (Chow et al. (1997) Behav Pharmacol 8(8):725-35; Hubbell et al. (1991) Alcohol 8(5):355-67; June et al. (1998) Alcohol Clin Exp Res 22(9):2174-85). Nalmefene acts on μ, δ, and κ receptors, providing more effective control of the non-μ receptor reinforcing effects of drinking than naltrexone, which primarily blocks μ receptors (Tabakoff and Hoffman (1983) Life Sci 32(3):197-204; Michel et al. (1985) Methods Find Exp Clin Pharmacol 7(4):175-77). Thus, nalmefene provides pharmacological and clinical advantages over naltrexone for the treatment of alcohol dependence. (Mason et al. (1999) Arch Gen Psychiatry 56(8):719-24)

Nalmefene has shown efficacy in two U.S. clinical studies (Mason et al. (1999), supra; Mason et al. (1994) Alcohol Clin Exp Res 18(5):1162-67). In a U.S. double blind, placebo-controlled study, 105 alcoholic patients who had been abstinent for two weeks received either 20 or 80 mg/day nalmefene orally, in conjunction with cognitive behavioral therapy. Fewer patients receiving nalmefene relapsed to heavy drinking (defined as ≧6 drinks per day for men and ≧4 drinks per day for women) over the twelve-week study period versus placebo. One-third of the nalmefene patients did relapse, but they had significantly fewer heavy drinking episodes than relapsing patients receiving placebo. There was a significant decrease at the first weekly study visit in percentage of nalmefene-treated patients reporting any heavy drinking days. The number of abstinent days and self-reported craving were the same in treated and control groups. Transient nausea was observed in the nalmefene-treated patients, although no serious adverse events occurred (Mason et al. (1999), supra). An earlier pilot study also reported a significantly lower rate of relapse as well as a greater increase in the number of abstinent days per week with 40 mg oral nalmefene, when compared with placebo or 10 mg nalmefene in 21 alcohol-dependent patients. Both 40 and 10 mg doses significantly decreased the number of drinks per drinking day (Mason et al. (1994), supra).

The clinical benefits of a long-term delivery system for treatment of alcoholism is illustrated by various studies that have used depots and implants. Disulfuram has been administered via subcutaneous implantation for treatment of alcoholism. Six studies showed inconsistent results but positive evidence that disulfiram reduces alcohol dependence. (Johnsen et al. (1987) Br J Addict 82(6):607-13; Johnsen and Morland (1991) Alcohol Clin Exp Res 15(3):532-36; Whyte and O'Brien (1974) Br J Psychiatry 124:42-44; Wilson et al. (1976) Br J Psychiatry 192:277-80; Wilson et al. (1978) J Stud Alcohol 39(5):809-19; Wilson et al. (1980) J Stud Alcohol 41(5):429-36). Naltrexone implants have been utilized for analgesia and opioid detoxification (Misra and Pontani (1981) NIDA Res Monogr 28:254-64; Schwope et al. (1975) NIDA Res Monogr 4:13-8; Yoburn et al. (1986) J Pharmacol Exp Ther 237(1):126-30). Complications have included pulmonary edema, prolonged withdrawal, drug toxicity, and withdrawal from cross-addiction to alcohol and benzodiazepines. (Hamilton et al. (2002) Acad Emerg Med 9(l):63-68)

Once-monthly depots of naltrexone have also been studied. Clinical studies have shown a significantly lower percentage of heavy drinking days in depot-treated patients (in combination with psychotherapy), versus patients receiving placebo plus therapy. The drawbacks of depot strategy include: (1) irritation observed with depots has been a limiting factor in clinical trials; (2) the irreversible nature of depots is a safety issue with respect to the irritation observed after injection, and allows less flexibility for dosing regimens; and (3) the once-monthly dosing regimen of a depot does not completely address the compliance issues associated with treatment of a chronic disease such as alcoholism.

There is a need for an improved method of long-term delivery of pharmaceuticals for treatment of alcoholism. A long-term method for continuous administration of nalmefene, which results in fewer adverse side effects than naltrexone or sulfuram, would be beneficial for treatment of alcoholism.

Nalmefene has also been shown to be effective for treatment of other conditions, such as, for example, nicotine dependence, impulse control disorders, for example pathological gambling, interstitial cystitis, narcotic overdose, pruritis, for example associated with cholestasis, and epidural-induced side effects, and for reversal of opioid sedation and reduction of food intake. An improved method for administering nalmefene for any of these indications, without the peaks and troughs associated with other means of administration and with improved patient compliance due to continuous dosing, would be desirable.

### BRIEF SUMMARY OF THE INVENTION

The invention provides compositions (ie., implantable polymeric devices), methods, and kits for treatment of alcoholism or nicotine dependence, or another condition for which nalmefene administration is therapeutically beneficial.

In one aspect, the invention provides an implantable device for administration of nalmefene to a mammal in need thereof, which includes nalmefene encapsulated in a biocompatible, nonerodible polymeric matrix. After subcutaneous implantation in a mammal, an implantable device of the invention releases nalmefene continuously in vivo through pores that open to the surface of the matrix at a rate that results in a plasma nalmefene level of at least about 0.01 ng/ml at steady state. In some embodiments, an implantable device of the invention includes ethylene vinyl acetate (EVA) as a biocompatible, nonerodible polymer for formation of the polymeric matrix. In one embodiment, the vinyl acetate content of EVA used for preparation of the polymeric matrix is often about 33%. In various embodiments, the nalmefene content in an implantable device of the invention is about 0.01 to about 90%, or any of at least about 0.01, 0.05, 1, 5, 10, 20, 50, 65, 70, 75, 80, 85, or 90%. Implantable devices often release nalmefene continuously in vivo for at least about 2 weeks, or 1, 3, 6, 9, 12, 15, 18, 21, or 24 months. In some embodiments, implantable devices of the invention are produced using an extrusion process to produce devices with dimensions of about 2 to about 3 mm in diameter and about 2 to about 3 cm in length, although other shapes and sizes are contemplated and are within the skill of the art. Generally, an implantable device of the invention releases nalmefene at a rate of about 0.01 to about 10 mg/day at steady state in vitro or in vivo. In one embodiment, the implantable devices release nalmefene at a rate of at least about 0.01 mg/day. In some embodiments, an implantable device of the invention include a difflusional barrier. In one embodiment, the difflusional barrier includes EVA, and optionally further includes nalmefene, for example EVA loaded with 10 or 20% nalmefene by weight.

In another aspect, the invention provides a method for administration of nalmefene to a mammal in need thereof. Methods of the invention include subcutaneous administration of at least one implantable device as described above. In some embodiments, the methods include subcutaneous implantation of a multiplicity of the devices. In one embodiment, the device or devices release nalmefene at a steady state level that is therapeutically effective for treatment of alcoholism in an individual in need of treatment. In another embodiment, the device or devices release nalmefene at a steady state level that is therapeutically effective for treatment of nicotine addiction. Often, a therapeutically effective steady state plasma level is at least about 0.01 ng/ml. Typically, each device, or the combination of a multiplicity of devices, continuously releases at least about 0.01 ng/ml at steady state. Generally, each device, or the combination of a multiplicity of devices, releases nalmefene at a steady state rate of at least about 0.01 mg/day in vitro or in vivo. In various embodiments, one or a multiplicity of devices is subcutaneously implanted in an individual on the upper arm, the back, and/or the abdomen.

In another aspect, the invention provides a kit comprising at least one implantable device as described above and instructions for use in a method of administration of nalmefene to a mammal in need thereof. In some embodiments, kits of the invention include a multiplicity of individual nalmefene-containing implantable devices. In one embodiment, a kit is provided for treatment of alcoholism. In another embodiment, a kit is provided for treatment of nicotine dependence.

### BRIEF DESCRIPTION OF THE DRAWINGS

FIG. 1 depicts in vitro release of nalmefene from extruded EVA-coated nalmefene-containing implants.

FIG. 2 depicts in vivo release of nalmefene in rats implanted with one or three EVA-coated nalmefene-loaded implantable devices.

### DETAILED DESCRIPTION OF THE INVENTION

The invention provides a biocompatible, nonerodible polymeric device, which permits controlled, sustained release of nalmefene over extended periods of time when implanted subcutaneously in an individual in need of treatment.

Continuous release of a compound in vivo over an extended duration may be achieved via implantation of a device containing the compound encapsulated in a nonerodible polymeric matrix. Examples of implantable, nonerodible polymeric devices for continuous drug release are described in, e.g., U.S. Pat. Nos. 4,883,666, 5,114,719, and 5,601,835. Implantation of the device and extended release of nalmefene improves compliance with dosing regimens, eliminating the need for repeated injections or ingestion of pills or tablets. An implantable, sustained-release device according to the present invention also permits achievement of more constant blood levels of nalmefene than injectable or oral dosage forms, thereby minimizing side effects and improving therapeutic effectiveness.

Devices of the invention include one or more non-bioerodible polymers. Such polymers release compounds at linear rates for extended time periods of several months or longer, in contrast to bioerodible polymers, which do not exhibit linear release kinetics due to formation of channels in the matrix as it erodes, resulting in increased release rates over time. The present invention includes a biocompatible, nonerodible polymer that exhibits generally linear release kinetics for nalmefene in vivo, after an initial burst.

Implantable Polymeric Devices

The invention includes implantable devices for administration of nalmefene to an individual in need thereof. Implantable devices of the invention contain nalmefene encapsulated in a polymeric, nonerodible matrix. As used herein, “nalmefene” refers to nalmefene and pharmaceutically acceptable salts thereof, such as for example, nalmefene HCl. Incorporation of nalmefene into the polymeric matrix causes the formation of a series of interconnecting channels and pores that are accessible to the surface for release of the drug. Where appropriate, a coating that is impermeable to the drug is placed over at least a portion of the device to further regulate the rate of release. Often, because nalmefene is highly soluble in aqueous environments, a difflusional barrier is added to the outer surface of the implantable devise to achieve a lower release rate in vivo. Examples of coating compositions include EVA or nalmefene-loaded EVA. For example, EVA loaded with about 10 or 20% nalmefene by weight may be used.

When implanted subcutaneously, devices of the invention continuously release nalmefene for an extended period of time with a pseudo or near zero order release rate. After an initial burst following implantation, release rates are typically within about 10-20% of the steady state average.

In some embodiments, the initial burst of nalmefene released in vivo after implantation is reduced or minimized by prewashing the implantable devices before implantation to remove surface nalmefene. Prewashing may be performed in any solution in which nalmefene is soluble, for example ethanol or normal saline, often for about 30 minutes.

As used herein, “nonerodible matrix” refers to a polymeric carrier that is sufficiently resistant to chemical and/or physical destruction by the environment of use such that the matrix remains essentially intact throughout the release period. The polymer is generally hydrophobic so that it retains its integrity for a suitable period of time when placed in an aqueous environment, such as the body of a mammal, and stable enough to be stored for an extended period before use. The ideal polymer must also be strong, yet flexible enough so that it does not crumble or fragment during use. Nonerodible matrices remain intact in vivo for extended periods of time, typically months or years. Drug molecules encapsulated in the matrix are released over time via diffusion through channels and pores in a sustained and predictable manner. The release rate can be altered by modifying the percent drug loading, porosity of the matrix, structure of the implantable device, or hydrophobicity of the matrix, or by adding a hydrophobic coating to the exterior of the implantable device.

Typically, ethylene vinyl acetate copolymer (EVA) is used as the polymeric matrix, but other nonerodible materials may be used. Examples of other suitable materials include silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, and combinations thereof.

Implantable devices of the invention are typically formulated with nalmefene loading of at least about 0.01%, often about 0.01 to about 90%. Devices are often formulated as compositions that include a polymeric matrix that includes EVA (33% acetate) and any of at least about 0.01, 0.05, 0.1, 0.5, 1, 5, 10, 20, 50, 65, 70, 75, 80, 85, or 90% nalmefene. Devices may be produced using an extrusion process, wherein ground EVA is blended with nalmefene, melted, and extruded into rod-shaped structures. Rods are cut into individual implantable devices of the desired length, packaged, and sterilized prior to use. Other methods for encapsulating therapeutic compounds in implantable polymeric, nonerodible matrices are well known to those of skill in the art. Such methods include, for example, solvent casting (see, e.g., U.S. Pat. Nos. 4,883,666, 5,114,719, and 5,601,835). A skilled artisan would be able to readily determine an appropriate method of preparing such an implantable device, depending on the shape, size, drug loading, and release kinetics desired for a particular type of patient or clinical indication.

Devices of the invention are suitable for sustained release of nalmefene for treatment of alcoholism or another condition for which administration of nalmefene is therapeutically beneficial, such as, for example, treatment of nicotine dependence. Other examples of uses for devices of the invention include treatment of impulse control disorders, for example pathological gambling, interstitial cystitis, narcotic overdose, pruritis, for example associated with cholestasis, reversal of opioid sedation, treatment of epidural-induced side effects, and reduction of food intake.

As used herein, “sustained release” refers to the release of nalmefene such that the blood concentration remains within the therapeutic range but below toxic levels for an extended duration. Devices of the invention generally exhibit near zero-order pharmacokinetics in vivo, similar to kinetics achieved with an IV drip, but without the need for external medical equipment and personnel associated with intravenous methods. Generally, after implantation, the devices release therapeutically effective amounts of nalmefene for periods of several months up to one year or longer.

Multiple implantable devices may be used, or the size and shape of the devices may be modified, to achieve a desired overall dosage. Implantable devices are often about 0.5 to about 10, more often about 1.5 to about 5, most often about 2 to about 3 cm in length, and are often about 0.5 to about 7, more often about 1.5 to about 5, most often about 2 to about 3 mm in diameter. The release rate of implantable devices may also be modified by changing the vinyl acetate content in the EVA polymer matrix. The vinyl acetate content is often about 2 to about 40, more often about 10 to about 35, most often about 30 to about 35% by weight. In one embodiment, the vinyl acetate content is about 33% by weight. The release rate may also be modified by coating the exterior surface of the implant with a difflusional barrier, such as an erodible or non-erodible polymer, for example EVA. Often, the surface is coated with about 25 weight percent EVA. In one embodiment, the diffusional barrier contains nalmefene, e.g., nalmefene-loaded EVA. The diffusional barrier may include, for example, any of the polymers listed in U.S. Pat. Nos. 4,883,666, 5,114,719, or 5,601,835.

Methods of the Invention

The invention provides methods for administration of nalmefene to an individual in need thereof. Nalmefene may be administered to an individual in accordance with the methods of the invention for treatment of a condition such as alcoholism, nicotine dependence, or another condition for which administration of nalmefene is therapeutically beneficial, such as those listed above.

In one embodiment, nalmefene is administered according to the methods of the invention for treatment for alcoholism. As used herein, “alcoholism” refers to a primary, chronic disease with genetic, psychosocial, and environmental factors influencing its development and manifestations. The disease is often progressive and fatal. It is characterized by impaired control over drinking, preoccupation with the drug alcohol, use of alcohol despite adverse consequences, and distortions of thinking, most notably denial. Each of these symptoms may be continuous or periodic.

In another embodiment, nalmefene is administered according to the methods of the invention for treatment of nicotine dependence.

Methods of the invention include subcutaneous administration of one or more polymeric implantable devices which include nalmefene encapsulated within a biocompatible, nonerodible polymeric matrix, e.g., EVA, and release of nalmefene in a controlled manner over an extended period of time through multiple pores that open to the surface of the implantable device(s). Often, implantable devices are produced via an extrusion process, as described above.

Implantable devices are administered by subcutaneous implantation to an individual in need of treatment with nalmefene. As used herein, “individual” refers to a mammal, such as a human in need of treatment for alcoholism, nicotine dependence, or another condition for which administration of nalmefene is therapeutically beneficial. Generally, implantable devices are administered by subcutaneous implantation at sites including, but not limited to, the upper arm, back, or abdomen of an individual. Other suitable sites for administration may be readily determined by a medical professional. Multiple implantable devices may be administered to achieve a desired dosage for treatment.

Typically, an implantable device or a multiplicity of devices is administered that will release nalmefene at a rate that will maintain a therapeutically effective plasma level for an extended period of time of at least about 2 weeks, or 1, 3, 6, 9, 12, 15, 18, 21, or 24 months. Often, the duration of implantation, with continuous release of nalmefene, is from about 3 months to about 2 years, about 3 months to about 1 year, about 3 months to about 9 months, or about 3 months to about 6 months.

The desired dosage rate will depend upon factors such as the underlying condition for which nalmefene is being administered, and the physiology of a particular patient, but will be readily ascertainable to physicians. Nalmefene is desirably released from one or a multiplicity of implanted devices at a rate that maintains plasma levels of the drug at a therapeutically effective level. Maintenance of nalmefene at a fairly constant plasma level often permits dosing at a lower level than with other therapies, such as oral administration.

As used herein, “therapeutically effective amount” or “therapeutically effective level” refers to the amount of nalmefene that will render a desired therapeutic outcome, i.e., a level or amount effective to reduce or alleviate symptoms of the condition for which nalmefene is administered. For example, a positive therapeutic outcome for treatment of alcoholism may include a decrease in relapse rate and increase in time to first relapse, increase in abstinence and number of abstinent days, decrease in alcohol consumption and number of drinks per day, and decrease in craving for alcohol. An amount that is “therapeutically effective” for a particular patient may depend upon such factors as a patient's age, weight, physiology, and/or the particular symptoms or condition to be treated, and will be ascertainable by a medical professional. When multiple devices are administered, the combination of the devices releases nalmefene at a rate that will achieve a therapeutically effective plasma level.

A therapeutically effective plasma level for treatment of alcoholism is often about 0.01 to about 70, about 0.05 to about 50, about 0.1 to about 25, or about 1 to about 10 ng/ml. Often, sustained release at this dosage rate occurs for about 2 weeks to about 1 year or longer (e.g., at least about 3, 6, 9, 12, 15, 18, 21, or 24 months). In various embodiments, an implantable device of the invention may release nalmefene in vivo at a rate that results in a steady-state plasma level of at least about 0.01, 0.05, 0.1, 0.5, 1, 2, 3, 4, 5, 10, 20, 30, 40, 50, 60, or 70 ng/ml. Typically, the release rate of nalmefene used for treatment of alcoholism is from about 0.01 to about 10 mg/day/implant.

In some embodiments, nalmefene is administered via implantable devices of the invention for treatment of alcoholism, in conjunction with other therapies including but not limited to brief intervention, community reinforcement, motivational enhancement, family therapy, social skills training, cognitive therapy, biofeedback, detoxification, electrical stimulation, aversion therapy stress management, antidepressants, hypnosis, acupuncture, alcoholics anonymous 12 step program, psychotherapy, tobacco cessation, GABA agonists, or opiate antagonists.

In methods for treatment of nicotine dependence, one or a multiplicity of nalmefene-containing implantable devices, as described above, are implanted in an individual in need of treatment, such that total release of nalmefene at steady state is about 0.01 to about 10 mg/day, and the steady state plasma level is about 0.01 to about 100 ng/ml, about 0.05 to about 50, about 0.1 to about 25, or about 1 to about 10 ng/ml, or at least about at least about 0.01, 0.05, 0.1, 0.5, 1, 2, 3, 4, 5, 10, 20, 30, 40, 50, 60, 70, 80, 90, or 100 ng/ml for at least about 2 weeks to about 1 year or longer (e.g., at least about 3, 6, 9, 12, 15, 18, 21, or 24 months).

It is anticipated that the implantable devices of the invention will alleviate compliance difficulties, as described above. In methods of the invention, long term continuous release of nalmefene generally reduces or eliminates the peaks and troughs of blood concentration of nalmefene associated with other formulations such as oral or injectable dosage forms, which often permits dosing at a lower level than traditional treatment regimens. This often reduces or alleviates adverse side effects associated with higher dosages.

Kits

The invention also provides kits for use in treatment of alcoholism, nicotine dependence, or another condition for which nalmefene administration is therapeutically beneficial, as described above. The kits contain at least one implantable, nonerodible device of the type herein described, capable of delivering long-term therapeutic levels of nalmefene, in suitable packaging, along with instructions providing information to the user and/or health care provider regarding subcutaneous implantation and use of the system for treating a condition for which nalmefene administration is therapeutically beneficial, such as, for example, alcoholism or nicotine dependence. Kits may also include literature discussing performance of the implantable devices of the invention.

Kits include a delivery system, i e., one or a multiplicity of implantable devices, capable of providing sustained release of therapeutic levels of nalmefene for at least about 2 weeks, often at least about 3 months. In kits of the invention, an implantable device or devices may be preloaded into an apparatus or apparatuses suitable for subcutaneous implantation of the device(s) into a patient, such as, for example, a syringe or trocar. Kits may also contain one or more oral dosage forms of nalmefene for titration of the nalmefene dose.

Kits for treatment of alcoholism typically contain a polymeric, nonerodible delivery system capable of continuously releasing nalmefene at a rate sufficient to achieve a therapeutically effective nalmefene plasma level, often about 0.01 to about 70, about 0.05 to about 50, about 0.1 to about 25 ng/ml, or about 1 to about 10 ng/ml, for at least about 3 months. In various embodiments, a delivery system is capable of releasing nalmefene in vivo at a rate that results in a steady-state plasma level of at least about 0.01, 0.05, 0.1, 0.5, 1, 2, 3, 4, 5, 10, 20, 30, 40, 50, 60, or 70 ng/ml. Often, sustained release at this dosage rate occurs for at least about 3 months to about 1 year or longer (e.g., at least about 3, 6, 9, or 12, 15, 18, 21, or 24 months). Kits of the invention may include a delivery system capable of releasing about 0.01 to about 10 mg/day nalmefene in vitro or in vivo.

Kits for treatment of nicotine dependence typically contain a delivery system capable of continuous nalmefene release at a steady-state level of 0.01 to about 100 ng/ml, about 0.05 to about 50, about 0.1 to about 25 ng/ml, or about 1 to about 10 ng/ml, or at least about 0.01, 0.05, 0.1, 0.5, 1, 2, 3, 4, 5, 10, 20, 30, 40, 50, 60, 70, 80, 90, or 100 ng/ml.

### EXAMPLES

The following examples are intended to illustrate but not limit the invention.

### Example 1

### Preparation of Nalmefene Implants

Implantable devices were prepared using an extrusion process. Nalmefene HCl was dried at 115-118° C. under high vacuum. The final moisture content of the nalmefene was 0.3870%. Moisture content was determined by thermal gravimetric analysis (TGA). Extrusion was performed using a blend of 65% nalmefene and 35% EVA (33% vinyl acetate). The processing conditions that were used are shown in Table 1.

TABLE 1
Conditions for Extrusion of Nalmefene Implants

| | |
|---|---|
| Augur rate | ˜71-72 rpm |
| Amps | ˜1.36 |
| Temperatures: | |
| Zone 1 | ˜110.5° C. |
| Zone 2 | ˜117.8° C. |
| Zone 3 | ˜110.5° C. |
| Zone 4 | ˜113.3° C. |

The extruded fiber was cut into 27 mm implants. These implants were coated using a 0.1% solution of 33% EVA dissolved in methylene chloride using a fluid-bed coater. The coating conditions were as shown in Table 2.

TABLE 2
Conditions for Spray Coating

| | |
|---|---|
| Inlet Temperature (° C.) | ˜32.2-33.3 |
| Outlet Temperature (° C.) | ˜22.2-23.3 |
| Fluidizing Air Flow | ˜0.80-0.75 |
| Filter Pressure (psi) | ˜12.5 |
| Lift Cylinder Pressure (psi) | >60 |
| Atomizing Air Flow (psi) | ˜5-7-6 |
| Panel Purge Volume (SCFH) | ˜20 |

The implants were packaged and sterilized by gamma radiation (2.5 mrads).

### Example 2

### Characterization of Extruded Implantable Devices

Extruded rods prepared as described above were characterized for total drug load and for rate of drug release.

Photomicrography

The surface and interior morphology of implants prepared as in Example 1 were examined using scanning electron microscopy (SEM). Implants were fractured cryogenically to expose the interior of the implant. Photomicrographs were taken to show one image of the microstructure of the lateral surface of the implant and one image of a cross section. From the SEM micrographs, the distribution of nalmefene and the coating looked very homogeneous.

Assessment of Drug Loading

The nalmefene content in the implants was determined by extracting the nalmefene with methylene chloride and quantitating the nalmefene using an HPLC method. The dimensions, weight, and nalmefene content of the implants is presented in Table 3.

TABLE 3
Nalmefene HCl/EVA Formulation

| | | Wt % (Nalmefene |
|---|---|---|
| Composition | Dimensions | HCl Content) |
| 35/65 Nalmefene/EVA | Diameter: 3 mm | 42% (73 mg) |
| | Length: 27 mm | |
| | Weight: 174 mg | |

Assessment of in Vitro Drug Release

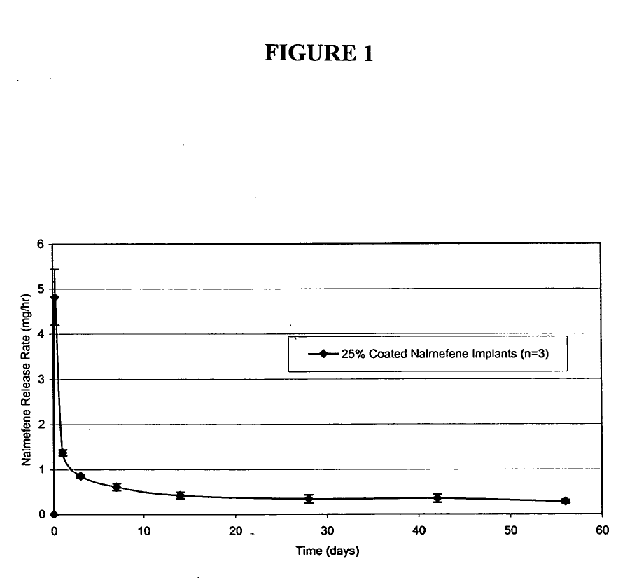

The in vitro release rate of nalmefene from the implants was determined by placing the implants in amber bottles containing 100 ml of normal saline. The sample bottles were placed in a 37° C. water bath agitating at 50 rpm. 100 μl sample aliquots were taken at various time points and replaced with fresh normal saline. The collected samples were analyzed for nalmefene HCl at each time point. The in vitro release studies showed that a steady state release rate was gradually attained after an initial burst (FIG. 1). The total percent of nalmefene release from the implants over 56 days was 30.4%. This study indicates that nalmefene can be released from the implantable devices at a controlled rate over an extended period of time.

### Example 3

### In Vivo Evaluation of Nalmefene Loaded Implantable Devices

Implants were prepared by extrusion of a 30:70 blend of EVA copolymer (33% vinyl acetate) and nalmefene HCl at an elevated temperature, yielding filaments with a 2.5 mm diameter, from which 2.6 cm implants were cut. The surface of the implants was coated with an EVA suspension (14 wt % EVA in water with sodium lauryl sulfate) using a Wurster fluidized bed coater to produce a 25 wt % coating. Implants were sterilized with γ-radiation. In vitro release of nalmefene from coated and uncoated implants, both including 70% nalmefene hydrochloride, was determined by release into 100 ml of saline at 37° C., followed by HPLC analysis. The in vitro drug release from uncoated implants was 26-52 mg/day. Coating the surface of the implants with 25 wt % EVA reduced the release rate to 0.286-0.607 mg/day. Gamma sterilization of the implants had no effect on the release rates.

Wistar-derived rats were surgically implanted with either 1 (n=8) or 3 (n=8) implants containing 73 mg of nalmefene per implant. Implants were placed subcutaneously on the back of the animal parallel to the spine. Plasma samples were taken from the tail vein before implant, and after implantation at 6 and 12 hours on day 1, every 48 hours until day 7, weekly until week 12 and then every 2 weeks until the end of the study at 24 weeks. Three animals from each group were terminated at 12 weeks, and the implants were explanted for content analysis. The animals were euthanized, and the skin along the back was resected to visualize the implants. The implants were photographed, removed, and analyzed by HPLC. The remaining animals were maintained until 24 weeks, at which time three animals from each group were terminated in the same manner. The remaining two animals from each group were explanted under anesthesia, and plasma samples taken at hours 3, 6, 9, 12, 24, and 48, to obtain elimination pharmacokinetic data. These animals were terminated at the end of 48 hours.

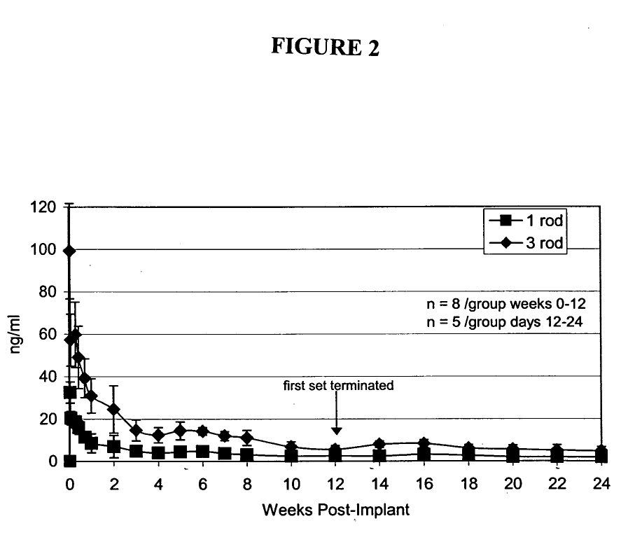

FIG. 2 shows the mean nalmefene plasma levels of each group throughout the course of the study. Plasma nalmefene levels from the animals with three implants were approximately three times higher than those of the animals with one implant at all time points. Two plasma level phases were observed, a “burst” phase of high levels that dropped by three weeks post-implantation, followed by a sustained-release phase from 3-24 weeks, during which time the plasma concentrations were 3.2±0.6 ng/ml and 8.8±0.7 ng/ml for the groups with one and three implants, respectively. Nalmefene release was 0.23±0.05 mg/implant/day. The elimination phase, monitored in four animals (two per group), showed plasma nalmefene levels below quantifiable limits (0.05 ng/ml) by six hours post-explantation.

During the “burst,” plasma concentrations reached 33 ng/ml for the one-implant group and 90 ng/ml for the three-implant group, approximately 10 times the plasma levels during sustained release. Approximately 38% of nalmefene release occurs during the first three weeks, while the remaining 62% is released during the 21 week sustained-release period. At the end of nearly 6 months, approximately 25% of the initial drug remained in the implants.

Results from this study indicate that nalmefene implants can provide sustained plasma levels of the drug for 6 months. Macroscopic examination of all implant sites showed no irritation. No adverse effects were observed for the duration of the study.

### Example 4

### Preparation and Evaluation of Implantable Devices Coated with Nalmefene-loaded EVA

Materials

Poly (ethylene-co-vinyl acetate) (EVA) pellets (33 wt % vinyl acetate) were obtained from Aldrich. Nalmefene hydrochloride was obtained from Diosynth.

Methods

Cryogenic grinding of EVA

The particle size of the EVA was reduced prior to dry blending with the nalmefene. 530 g of EVA pellets was milled in a Retsch ZM 100 Ultra Centrifugal Mill (Glen Mills, Inc., Clifton, N.J.). The EVA was premixed with liquid nitrogen and then transferred to the grinding chamber of the mill, where it passed through a 0.5 mm screen at a speed of 18,000 rpm. The milled EVA was sieved with a 850 μm screen and particles that were less than 850 μm were dried under vacuum at room temperature for 3 days. The yield of milled EVA less than 850 μm was about 350 g.

Particle size reduction and drying of nalmefene hydrochloride

Three hundred grams of nalmefene hydrochloride was ground with a mortar and pestle to reduce the particle size and then sieved to collect particles between 53 and 180 μm. The sieved nalmefene hydrochloride was dried in a vacuum oven for about 12 hours at 118° C. Due to clumping of the nalmefene particles, the dried nalmefene was re-sieved to collect particles between 53 and 180 μm.

The moisture content of the nalmefene before and after drying was determined by thermal gravimetric analysis using a TA Instruments Thermogravimetric Analyzer. Nalmefene samples were heated from 20 to 120° C. at 5° C. per minute until equilibrated at 120° C. The temperature was then ramped to 214° C. at 2° C. per minute. The initial moisture content before drying was about 4.4% and after drying, the moisture content was reduced to about 0.03%.

The particle size of the nalmefene before and after sieving was determined using a Coulter LS 13,230 particle size analyzer. A solution of 0.1% Span 85/heptane was used to suspend the nalmefene particles for the particle size analysis. The mean particle size before sieving was 203.5 μm and the mean particle size after sieving was 99.87 μm.

Preparation of dry blends for extrusion

Nalmefene and EVA, prepared as described above, were combined in a screw-cap glass jar. The jar was sealed and inverted several times for 5 minutes while occasionally rotating the jar sideways until the components were uniformly mixed as indicated by visual appearance. The nalmefene/EVA blends were prepared inside a glove box under nitrogen to keep the nalmefene dry.

Preparation of coated nalmefene implant formulations

Coated implants were prepared using a two-step process. The core was first extruded as a monolithic rod using an RCP-0500 extruder. A coating was then applied separately by passing the rod through a heated die coating assembly containing the coating material.

A monolithic rod was prepared from a 75/25 nalmefene/EVA blend using an RCP-0500 extruder using process conditions as shown in Table 4.

TABLE 4
Conditions for Extrusion of Nalmefene Implants

| | |
|---|---|
| Extrusion Temperature | |
| Zone 1 | 99° C. |
| Zone 2 | 121° C. |
| Zone 3 | 116° C. |
| Zone 4 (Die) | 116° C. |
| Melt Temperature | 117° C. |
| Pressure | 800-1400 psi |
| Amps | 1.5-2.2 |
| Extruder Screw Speed | 0.1-1.9 rpm |
| Die Orifice | 4.0 mm |

Seven cm length samples were cut from the 75/25 nalmefene/EVA rod to prepare coated implants. A stainless steel die coating assembly with a 4.4 mm diameter orifice was preheated to about 127° C. and was then loaded with a coating material of a 10 or 20% nalmefene in EVA. Each implant was suspended on a needle and then passed through the orifice of the die coating assembly where it was coated with the molten coating material.

Coated implants were cooled to room temperature and then cut to lengths of 5.2 cm. The ends of the coated implants were sealed with the respective molten coating material.

Core loading determination procedure

Triplicate samples (20 to 40 mg) of implant formulations were placed in 50 ml screw-cap culture tubes. Five ml of methylene chloride was added to each sample. The tubes were sealed and sonicated for approximately 10 minutes, or longer if required for complete disintegration of the samples by visual inspection.

Forty ml of deionized water was added to each sample and vortexed vigorously for 60 seconds to extract the nalmefene from the methylene chloride suspension. The samples were permitted to stand at room temperature for approximately 1 hour with frequent vortexing. The samples were then permitted to stand at room temperature until the two layers separated. The upper layer (deionized water) from each sample was transferred to a 100 ml volumetric flask. Thirty ml of deionized water was added to each sample. Samples were then vortexed vigorously for 30 seconds. The tubes were then permitted to stand at room temperature until the two layers separated. The upper layer was combined with the upper layer from the previous extraction in the appropriate volumetric flask.

Each flask was diluted to volume with deionized water and mixed thoroughly. Approximately 1.5 ml of each sample was transferred into a 1.5 ml microcentrifuge tube and centrifuged for 5 minutes at 8,000 rpm to separate the two layers. Approximately 1 ml of each sample was transferred to an HPLC vial for analysis. Samples were diluted with deionized water as appropriate for keeping sample concentrations within the limits of the standard curve.

Triplicate control samples were prepared consisting of approximately 30 mg of nalmefene and 10 mg of EVA and processed as above.

In vitro release procedure

Coated implants were weighed and placed in clear glass bottles containing 100 ml of normal saline. The bottles were sealed with Teflon-lined screw caps and placed in a 37±2° C. shaking water bath and agitated at 50 rpm. Samples were removed for analysis after 15 minutes, 1, 2, and 5 hours, and 1, 2, 4, 7, 10, and 14 days. At each time point, a 2 ml aliquot was removed for analysis and replaced with 2 ml normal saline, except for the 4, 10, and 14 day time points, when the implants were transferred to bottles containing 100 ml of fresh normal saline. Samples removed for analysis were stored at 2-8° C. until analyzed by HPLC for nalmefene content.

Operating conditions for HPLC analysis were as shown in Table 5.

TABLE 5
Operating Conditions for HPLC Analysis of Nalmefene Content

| | |
|---|---|
| Mobile Phase | 30/70 vol/vol acetonitrile/(0.2% triethylamine in |
| | 0.05 M potassium phosphate monobasic, pH 4.2) |
| Flow Rate | 1.0 ml/min |
| Column | Symmetry C18, 5 μm particle size, 250 × 4.6 mm |
| Guard Column | Symmetry C18, 5 μm particle size, 3.9 × 20 mm |
| Detection | 270 nm |
| Injection Volume | 20 μl |
| Temperature | Ambient |
| Run Time | 10 min |
| Needlewash | Nanopure water |

Results

Nalmefene content of implant formulations

Nalmefene content in coated and uncoated implants was determined using the core loading determination procedure described above. Mean recoveries were 96, 90, and 101% of the theoretical loading for uncoated implants, coated implants with 10% nalmefene coating, and coated implants with 20% nalmefene coating, respectively. The mean recovery for nalmefene/EVA control samples was 97%.

In vitro nalmefene release

In vitro release of nalmefene from coated and uncoated implants was determined as described above.

By Day 14, uncoated implants released approximately 92% of the nalmefene core loading compared to approximately 33-36% for implants with a 10% nalmefene coating and approximately 65% for implants with a 20% nalmefene coating. With a low initial burst, the coated implants provided a steady release of nalmefene through Day 14.

Coated implant samples were sterilized by exposure to 2.5 (±10%) Mrads of gamma radiation. Very little difference in the release profiles was observed between the sterilized and unsterilized implant formulations containing the 10% nalmefene coating.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety.

1. An implantable device for administration of nalmefene to a mammal in need thereof, comprising nalmefene and a biocompatible, nonerodible polymeric matrix,
wherein said nalmefene is encapsulated within said matrix, and wherein when said implantable device is implanted subcutaneously in said mammal, said nalmefene is continuously released in vivo over a sustained period of time through pores that open to the surface of said matrix at a rate that results in a plasma level of at least about 0.01 ng/ml at steady state.

2. An implantable device according to claim 1, wherein the polymeric matrix comprises ethylene vinyl acetate copolymer (EVA).

3. An implantable device according to claim 2, wherein said EVA comprises about 33% vinyl acetate.

4. An implantable device according to claim 1, comprising about 0.01 to about 90% nalmefene.

5. An implantable device according to claim 1, further comprising a difflusional barrier.

6. An implantable device according to claim 5, wherein said diffusional barrier comprises EVA.

7. An implantable device according to claim 6, wherein said diffusional barrier comprises nalmefene.

8. An implantable device according to claim 1, wherein the sustained period of time is at least about 3 months.

9. An implantable device according to claim 1, wherein the implantable device is produced by an extrusion process.

10. An implantable device according to claim 9, comprising dimensions of about 2 to about 3 mm in diameter and about 2 to about 3 cm in length.

11. An implantable device according to claim 10, wherein said implantable device releases at least about 0.01 mg of nalmefene per day in vitro at steady state.

12. An implantable device for administration of nalmefene to a mammal in need thereof, comprising nalmefene and a biocompatible, nonerodible polymeric matrix,
wherein said nalmefene is encapsulated within said matrix, and
wherein when said implantable device is subcutaneously implanted in a mammal, said nalmefene is continuously released in vivo over a sustained period of time through pores that open to the surface of said matrix at a rate of at least about 0.01 mg of nalmefene per day at steady state.

13. An implantable device according to claim 12, wherein the polymeric matrix comprises EVA.

14. An implantable device according to claim 13, wherein said EVA comprises 33% vinyl acetate.

15. An implantable device according to claim 12, comprising about 0.01 to about 90% nalmefene.

16. An implantable device according to claim 12, further comprising a difflusional barrier.

17. An implantable device according to claim 16, wherein said difflusional barrier comprises EVA.

18. An implantable device according to claim 17, wherein said difflusional barrier comprises nalmefene.

19. An implantable device according to claim 12, wherein the sustained period of time is at least about 3 months.

20. An implantable device according to claim 12, wherein the implantable device is produced by an extrusion process.

21. A method for administration of a nalmefene to a mammal in need thereof, the method comprising administering at least one implantable device subcutaneously,
wherein each of said at least one implantable devices comprises nalmefene encapsulated within a biocompatible, nonerodible polymeric matrix,
wherein said nalmefene is continuously released in vivo from each of said at least one implantable devices over a sustained period of time through pores that open to the surface of said matrix at a rate that results in a plasma level of at least about 0.01 ng/ml at steady state.

22. A method according to claim 21, wherein said at least one implantable device comprises a multiplicity of individual implantable devices, and wherein the combination of said implantable devices continuously releases nalmefene in vivo over a sustained period of time at a rate that results in a plasma level of at least about 0.01 ng/ml at steady state.

23. A method according to claim 21, wherein the polymeric matrix comprises EVA.

24. A method according to claim 21, wherein said EVA comprises about 33% vinyl acetate.

25. A method according to claim 21, wherein each of said at least one implantable devices comprises at about 0.01 to about 90% nalmefene.

26. A method according to claim 21 for treatment of alcoholism.

27. A method according to claim 21 for treatment of nicotine dependence.

28. A method according to claim 21, wherein the sustained period of time is at least about 3 months.

29. A method according to claim 21, wherein each of said at least one implantable devices is produced by an extrusion process.

30. A method according to claim 29, wherein each implantable device comprises dimensions of about 2 to about 3 mm in diameter and about 2 to about 3 cm in length.

31. A method according to claim 30, wherein each implantable device releases at least about 0.01 mg of nalmefene per day in vitro.

32. A method according to claim 21, wherein each of said at least one implantable devices is subcutaneously implanted at a site selected from the group consisting of the upper arm, the back, and the abdomen.

33. A kit comprising at least one implantable device comprising nalmefene encapsulated within a biocompatible, nonerodible polymeric matrix, wherein when said at least one implantable device is implanted subcutaneously in a mammal, said nalmefene is continuously released in vivo from each of said at least one implantable devices over a sustained period of time through pores that open to the surface of said matrix at a rate that results in a plasma level of at least about 0.01 ng/ml at steady state, and instructions for use in a method of administration of nalmefene to a mammal in need thereof.

34. A kit according to claim 33, wherein said at least one implantable device comprises a multiplicity of individual implantable devices, and wherein when the combination of said implantable devices is implanted subcutaneously in a mammal, said implantable devices continuously release nalmefene in vivo over a sustained period of time at a rate that results in a plasma level of at least about 0.01 ng/ml at steady state.

35. A kit according to claim 33, wherein each of said implantable devices releases nalmefene at a rate of at least about 0.01 mg per day in vitro.

36. A kit according to claim 33, wherein each of said implantable devices comprises EVA.

37. A kit according to claim 36, wherein said EVA comprises about 33% vinyl acetate.

38. A kit according to claim 33, wherein each of said implantable devices comprises about 0.01 to about 90% nalmefene.
